(19) United States
(12) Patent Application Publication
(10) Pub. No.: US 2025/0186981 A1
(43) Pub. Date: Jun. 12, 2025

(54) CATALYST COMPOSITION FOR CYCLIC OLEFIN POLYMERIZATION, AND METHOD FOR PREPARING CYCLIC OLEFIN-BASED OLIGOMER OR OLEFIN-BASED POLYMER BY USING SAME

(71) Applicant: KYUNGPOOK NATIONAL UNIVERSITY INDUSTRY-ACADEMIC COOPERATION FOUNDATION, Daegu (KR)

(72) Inventors: Hyo Sun LEE, Daegu (KR); Hyeonuk YEO, Daegu (KR); Yerim CHO, Daegu (KR); Saira NAYAB, Daegu (KR)

(21) Appl. No.: 19/059,781

(22) Filed: Feb. 21, 2025

Related U.S. Application Data
(63) Continuation of application No. PCT/KR2023/010422, filed on Jul. 19, 2023.

(30) Foreign Application Priority Data
Aug. 25, 2022 (KR) 10-2022-0106900

(51) Int. Cl. B01J 31/18; B01J 31/02; B01J 31/22; C07C 2/46; C08F 32/08

(52) U.S. Cl. CPC B01J 31/1835; B01J 31/0279; B01J 31/0298; B01J 31/1815; B01J 31/2226; C07C 2/465; C08F 32/08; B01J 2231/10; B01J 2531/004; B01J 2531/828

(57) ABSTRACT

The present invention relates to a catalyst composition for cyclic olefin polymerization, and a method for preparing a cyclic olefin-based oligomer or a cyclic olefin-based polymer by using same, and, more specifically, to: a catalyst composition for cyclic olefin polymerization, the composition forming catalytically active species inside and outside the same reaction system during polymerization of cyclic olefin-based monomers, thereby enabling an oligomer or a polymer to be prepared from the cyclic olefin-based monomers; and a method for preparing a cyclic olefin-based oligomer or a cyclic olefin-based polymer by using same.

### CROSS-REFERENCE TO RELATED APPLICATIONS

This application is a Continuation of Application No. PCT/KR2023/010422, filed on Jul. 19, 2023, which in turn claims the benefit of Korean Patent Application No. 10-2022-0106900, filed on Aug. 25, 2022. The entire disclosures of all these applications are hereby incorporated by reference.

### TECHNICAL FIELD

The present disclosure relates generally to a catalyst composition for cyclic olefin polymerization, and a method for preparing a cyclic olefin-based oligomer or a cyclic olefin-based polymer by using the same. More particularly, the present disclosure relates to: a catalyst composition for cyclic olefin polymerization, the catalyst composition being capable of enabling a cyclic olefin-based oligomer or a cyclic olefin-based polymer to be prepared from a cyclic olefin-based monomer; and a method for preparing a cyclic olefin-based oligomer or a cyclic olefin-based polymer by using the same.

### BACKGROUND ART

Until now, inorganic materials such as silicon oxide and silicon nitride have been mainly used in the information and electronics industry. Along with an increasing demand for small-sized and highly efficient devices, demand for high-functionality new materials is increasing. As a material that can satisfy such high-performance characteristic requirements, there is growing interest in oligomers and polymers that have low dielectric constant and water absorption, excellent metal adhesion, mechanical strength, heat resistance and transparency, and high glass transition temperature (Tg>250° C.).

These oligomers or polymers can be used as electronic materials such as insulating films for semiconductors or TFT-LCDS, polarizer protective films, multichip modules, integrated circuits (ICS), printed circuit boards, encapsulating materials for electronic materials, or flat panel displays.

In particular, cyclic olefin-based polymers, which are polymers composed of cyclic monomers such as norbornene, have better transparency, heat resistance, and chemical resistance, and much lower birefringence and water absorption, compared with conventional olefin-based polymers, so they can be widely applied as optical materials for CDs, DVDs, or plastic optical fibers (POFs), information and electronic materials for capacitor films or low dielectrics, medical materials for low-absorbent syringes or blister packagings, etc.

As a catalyst used for polymerizing such cyclic olefin-based polymers, U.S. Pat. No. 5,705,503 discloses a method for polymerizing a norbornene-based monomer in the presence of a catalyst complex represented by [(Allyl)PdCl]2/AgSbF₆. However, since the catalyst to monomer ratio is 1:100 to 1:250 and the amount of catalyst used is excessive, a large amount of catalyst residue remains in a finally prepared polymer. Therefore, there is a possibility that the polymer may deteriorate due to thermal oxidation in the future, and light transmittance may also deteriorate.

Further, U.S. Pat. No. 6,455,650 discloses a method for polymerizing a norbornene-based monomer in the presence of a catalyst complex represented by [(R′)_{z}M(L′)ₓ(L″)_{y}]_{b}[WCA]_{d}. However, there is a problem in that it is not suitable for producing a polymer because the polymerization yield of the norbornene-based monomer is very low at 5%.

Meanwhile, U.S. Pat. Nos. 6,031,058 and 6,455,650 disclose methods for polymerizing cyclic olefins using methylaluminoxane (MAO) or organic aluminum as a cocatalyst. However, a separate step for converting a catalyst precursor to an activated catalyst is required, and there is a problem in that excessive use of expensive methylaluminoxane (MAO) or organic aluminum reduces industrial usability. Also, an obtained polymer becomes colored or problematic in its transparency.

Accordingly, there is a need for a catalyst system capable of polymerizing a cyclic olefin-based monomer in an economical manner without generating catalyst residues.

### DOCUMENTS OF RELATED ART

### Patent Documents

(Patent Document 1) U.S. Pat. No. 5,705,503 (registration date: 1998.01.06.)
(Patent Document 2) U.S. Pat. No. 6,455,650 (registration date: 2002.05.02.)
(Patent Document 3) U.S. Pat. No. 6,031,058 (registration date: 2000.02.29.)
(Patent Document 4) U.S. Pat. No. 6,455,650 (registration date: 2002.05.02.)

### DISCLOSURE

### Technical Problem

Accordingly, the present disclosure has been made keeping in mind the above problems occurring in the related art, and an objective of the present disclosure is to provide a catalyst composition for cyclic olefin polymerization, the catalyst composition being capable of enabling a catalyst to be prepared in situ as well as ex situ, thereby enabling a catalyst for cyclic olefin polymerization to be prepared simply and economically, and enabling a cyclic olefin-based oligomer or cyclic olefin-based polymer having high catalytic activity in cyclic olefin polymerization and a low residual amount of metal catalyst.

In addition, another objective of the present disclosure is to provide a method for preparing a cyclic olefin-based oligomer or a cyclic olefin-based polymer, the method being capable of polymerizing a cyclic olefin-based monomer in the presence of the above-described catalyst composition for cyclic olefin polymerization, thereby enabling a cyclic olefin-based oligomer or a cyclic olefin-based polymer with a low residual amount of metal catalyst to be prepared economically.

### Technical Solution

In order to accomplish the above objectives, according to one aspect of the present disclosure, there is provided a catalyst composition for cyclic olefin polymerization, the catalyst composition including: a palladium or cobalt-containing metal precursor compound; an amine compound represented by Formula 1 or 2 below; and a cocatalyst compound,

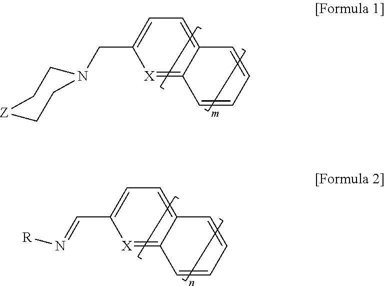

wherein in Formulae 1 and 2 above, X may be a carbon atom or a nitrogen atom, Z may be selected from the group consisting of an oxygen atom, a sulfur atom, and —(CH₂)—, R may be a hydrogen atom or a straight or branched alkyl group having 1 to 20 carbon atoms, and m and n may be the same or different and be each independently an integer of 0 to 3.

According to another aspect of the present disclosure, there is provided a catalyst composition for cyclic olefin polymerization, the catalyst composition including: a complex formed by a coordination bond between a palladium or cobalt-containing metal precursor compound and an amine compound represented by Formula 1 or 2 below; and a cocatalyst compound,

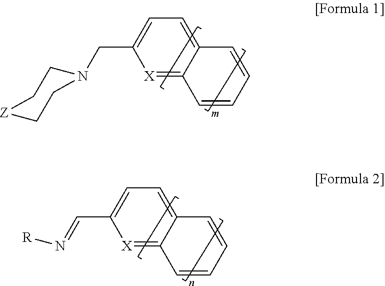

wherein in Formulae 1 and 2 above, X may be a carbon atom or a nitrogen atom, Z may be selected from the group consisting of an oxygen atom, a sulfur atom, and —(CH₂)—, R may be a hydrogen atom or a straight or branched alkyl group having 1 to 20 carbon atoms, and m and n may be the same or different and be each independently an integer of 0 to 3.

In a preferred embodiment of the present disclosure, in Formula 2, R may be a straight alkyl group having 1 to 7 carbon atoms.

In a preferred embodiment of the present disclosure, the amine compound represented by Formula 1 above may be a compound represented by any one of Formulae 1a, 1b, and 1c below,

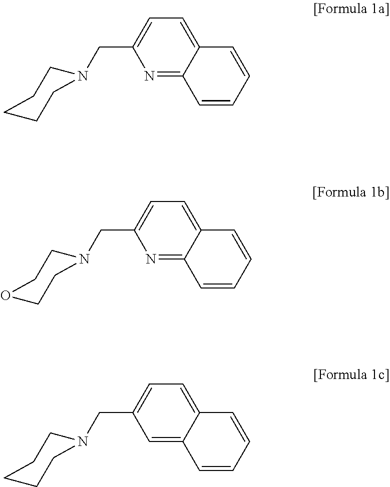 [SMILES: c1ccc2nc(CN3CCCCC3)ccc2c1 ; c1ccc2nc(CN3CCOCC3)ccc2c1 ; c1ccc2cc(CN3CCCCC3)ccc2c1]

In a preferred embodiment of the present disclosure, the amine compound represented by Formula 2 above may be a compound represented by Formula 2a below,

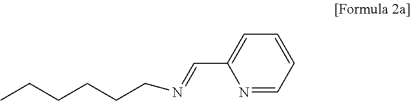

In a preferred embodiment of the present disclosure, the cocatalyst compound may be an organic boron compound.

In a preferred embodiment of the present disclosure, the organic boron compound may be tetrakis(pentafluorophenyl)borate.

In a preferred embodiment of the present disclosure, the amine compound represented by Formula 1 or 2 above may be contained in an amount of 1 to 5 moles per 1 mole of the palladium or cobalt-containing metal precursor compound.

In a preferred embodiment of the present disclosure, the cocatalyst compound may be contained in an amount of 2 to 10 moles per 1 mole of the palladium or cobalt-containing metal precursor compound.

In a preferred embodiment of the present disclosure, the complex may be a compound represented by any one of Formulae 3, 4, and 5 below,

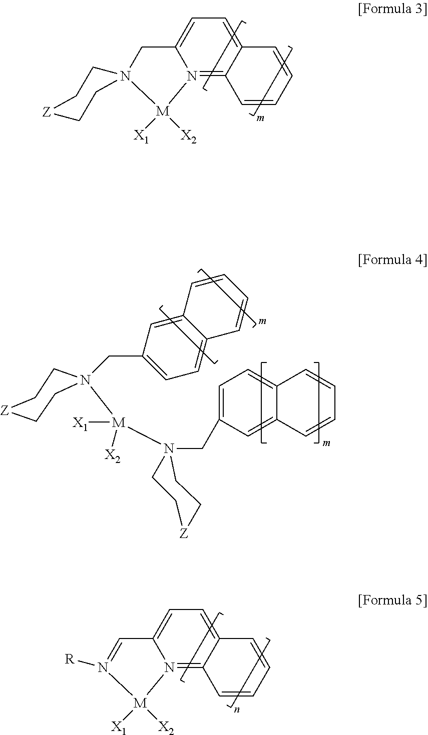

wherein in Formulae 3 to 5 above, M may be palladium or cobalt, Z may be the same or different and be independently selected from the group consisting of an oxygen atom, a sulfur atom, and —(CH₂)—, R may be a hydrogen atom or a straight or branched alkyl group having 1 to 20 carbon atoms, X₁ and X₂ may be the same or different and be each independently selected from the group consisting of an acetoxy group, a hydroxy group, an alkoxy group, and a halogen group, and m and n may be the same or different and be each independently an integer of 0 to 3.

According to another aspect of the present disclosure, there is provided a method for preparing a cyclic olefin-based oligomer, the method including: subjecting a cyclic olefin-based monomer to an oligomerization reaction in the presence of the catalyst composition.

In a preferred embodiment of the present disclosure, the cyclic olefin-based monomer may be a compound represented by Formula 10 below,

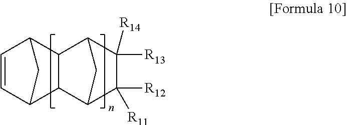

wherein in Formula 10 above, n may be an integer of 0 to 4, and R₁₁ to R₁₄ may be the same or different and be each independently selected from the group consisting of: a hydrogen atom; a halogen atom; a hydroxyl group; a carboxyl group; a straight or branched alkyl group having 1 to 20 carbon atoms; a cycloalkyl group having 3 to 12 carbon atoms; an aryl group having 6 to 20 carbon atoms; an alkoxy group having 1 to 10 carbon atoms; and an acyl group having 1 to 10 carbon atoms.

According to another aspect of the present disclosure, there is provided a method for preparing a cyclic olefin-based polymer, the method including: performing addition polymerization of a cyclic olefin-based monomer in the presence of the catalyst composition.

In a preferred embodiment of the present disclosure, the cyclic olefin-based monomer may be a compound represented by Formula 10 below,

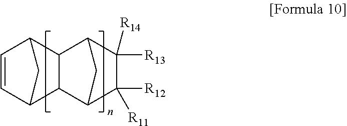

wherein in Formula 10 above, n may be an integer of 0 to 4, and R₁₁ to R₁₄ may be the same or different and be each independently selected from the group consisting of: a hydrogen atom; a halogen atom; a hydroxyl group; a carboxyl group; a straight or branched alkyl group having 1 to 20 carbon atoms; a cycloalkyl group having 3 to 12 carbon atoms; an aryl group having 6 to 20 carbon atoms; an alkoxy group having 1 to 10 carbon atoms; and an acyl group having 1 to 10 carbon atoms.

### Advantageous Effects

A catalyst composition for cyclic olefin polymerization according to the present disclosure contains a non-metallic compound as a cocatalyst compound, and enables a catalytically active species to be formed in situ as well as ex situ during polymerization of a cyclic olefin-based monomer, thereby reducing the amount of metal catalyst residue in a prepared cyclic olefin-based oligomer or cyclic olefin-based polymer. Therefore, it is possible to prepare of a high-quality oligomer or polymer having excellent physical properties such as optical properties and heat resistance without requiring a purification process, and eliminate a catalyst synthesis process or residual catalyst removal process, thereby providing an economical and environmentally friendly process.

### DESCRIPTION OF DRAWINGS

FIG. 1 is a view illustrating the X-ray structure of a palladium complex catalyst of Preparation Example 5 according to the present disclosure.

### MODE FOR INVENTION

Unless otherwise defined, all terms including technical and scientific terms used herein have the same meaning as commonly understood by one of ordinary skill in the art to which the present disclosure belongs. Generally, the nomenclature used herein is well known in the art and is the nomenclature normally used.

As used herein, when any part “includes” any element, this indicates that other elements are not excluded but may be further included unless otherwise specifically mentioned.

An aspect of the present disclosure relates to a catalyst composition for cyclic olefin polymerization, the catalyst composition including: a palladium or cobalt-containing metal precursor compound; an amine compound represented by Formula 1 or 2 below; and a cocatalyst compound.

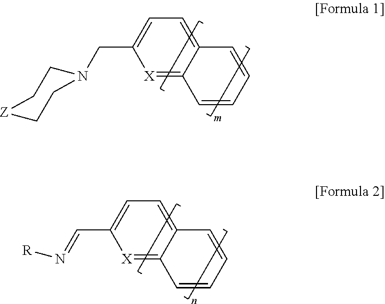

In Formulae 1 and 2 above, X is a carbon atom or a nitrogen atom, Z is selected from the group consisting of an oxygen atom, a sulfur atom, and —(CH₂)—, R is a hydrogen atom or a straight or branched alkyl group having 1 to 20 carbon atoms, and m and n are the same or different and are each independently an integer of 0 to 3. The metal precursor compound is a compound containing palladium or cobalt.

In addition, another aspect of the present disclosure relates to a catalyst composition for cyclic olefin polymerization, the catalyst composition including: a complex formed by a coordination bond between a palladium or cobalt-containing metal precursor compound and an amine compound represented by Formula 1 or 2 below; and a cocatalyst compound.

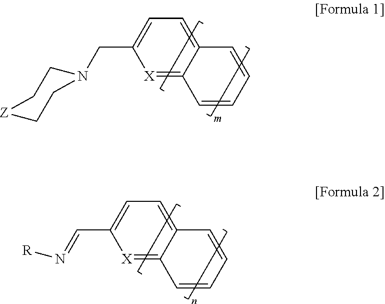

In Formulae 1 and 2 above, X is a carbon atom or a nitrogen atom, Z is selected from the group consisting of an oxygen atom, a sulfur atom, and —(CH₂)—, R is a hydrogen atom or a straight or branched alkyl group having 1 to 20 carbon atoms, and m and n are the same or different and are each independently an integer of 0 to 3.

More specifically, a catalyst composition for cyclic olefin polymerization according to an embodiment of the present disclosure includes: a palladium or cobalt-containing metal precursor compound; an amine compound represented by Formula 1 or 2 above; and a cocatalyst compound. The catalyst composition enables a complex to be instantly formed during a polymerization or oligomerization process of a cyclic olefin-based monomer, thereby producing a polymer or oligomer of the cyclic olefin-based monomer.

In addition, a catalyst composition for cyclic olefin polymerization according to another embodiment of the present disclosure includes a complex in which an amine compound represented by Formula 1 or 2 above is coordinately bonded to the palladium or cobalt-containing metal precursor compound; and a cocatalyst compound. The catalyst composition enables a cyclic olefin-based monomer to be polymerized or oligomerized at a high conversion rate.

In the present disclosure, the metal precursor compound is a compound containing palladium or cobalt, preferably palladium, which exhibits excellent catalytic activity in an oligomerization reaction or polymerization reaction of a cyclic olefin-based monomer. Any precursor such as a salt compound or complex of palladium or cobalt may be used without particular limitation. As for the salt compound, a salt compound that can be dissolved in an organic solvent may be used, and specifically, examples thereof include acetate, nitrate, sulfate, carbonate, hydroxide, halide, and hydrate thereof. As for the complex, examples thereof include an acetylacetonate complex, and a phosphine complex.

Meanwhile, in the present disclosure, the amine compound is an amine compound represented by Formula 1 or 2 above, and is a ligand compound that forms a complex in situ or ex situ by instantly coordinating the N position to the metal precursor compound added together during the oligomerization reaction or polymerization reaction of the cyclic olefin-based monomer.

Here, in Formula 1 above, X may be a carbon atom or a nitrogen atom, Z may be selected from the group consisting of an oxygen atom, a sulfur atom, and —(CH₂)—, preferably an oxygen atom or —(CH₂)—, m may be an integer of 0 to 3, and preferably m may be an integer of 1 to 3.

In addition, in Formula 2 above, X may be a carbon atom or a nitrogen atom, R may be a hydrogen atom or a straight or branched alkyl group having 1 to 20 carbon atoms, preferably a straight alkyl group having 1 to 7 carbon atoms, n may be an integer of 0 to 3, and preferably m may be an integer of 0 to 2.

Due to the abundant electron density of nitrogen atoms and phenyl groups, the amine compound may easily provide a lone pair of electrons to a ligand of palladium or cobalt, so it may be applied to the oligomerization reaction or polymerization reaction of the cyclic olefin-based monomer both in situ and ex situ, and may exhibit high reaction activity.

Specifically, an amine compound according to an embodiment of the present disclosure may be a compound represented by any one of Formulae 1a, 1b, 1c, and 2a below.

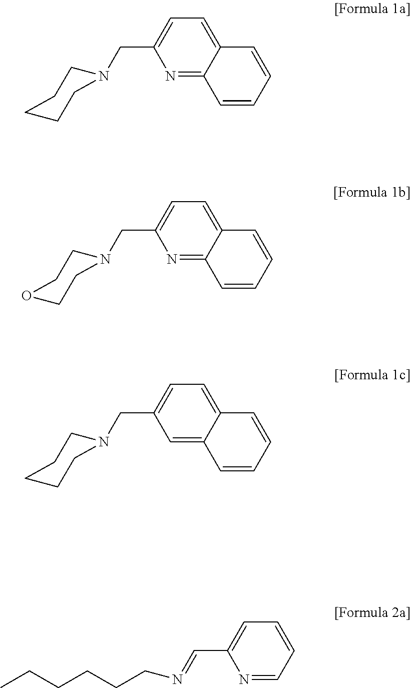 [SMILES: c1ccc2nc(CN3CCCCC3)ccc2c1 ; c1ccc2nc(CN3CCOCC3)ccc2c1 ; c1ccc2cc(CN3CCCCC3)ccc2c1 ; CCCCCC/N=C/c1ccccn1]

The amine compound may be contained in an amount of 1 to 5 mol, preferably 1 to 3 mol, and more preferably 1 mol, per 1 mol of the palladium or cobalt-containing metal precursor compound. When the above content ratio is satisfied, the N portion of a monodentate or bidentate ligand may be coordinated to the metal precursor compound to form a complex in a high yield either ex situ or in situ.

In the present disclosure, the complex is formed by coordinately bonding the amine compound represented by Formula 1 or 2 above to palladium or cobalt of the metal precursor compound, and may preferably include at least one of compounds represented by Formulae 3, 4, and 5 below.

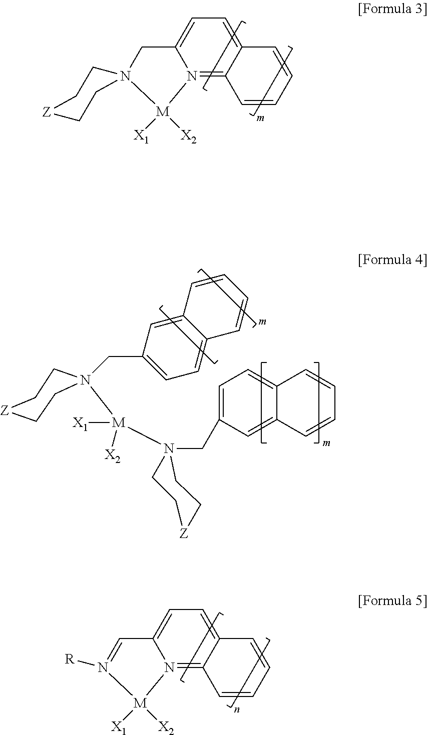

In Formulae 3 to 5 above, M, X, Z, R, m, and n are substantially the same as M, X, Z, R, m, and n described in Formulae 1 and 2, and X₁ and X₂ are the same or different and are each independently selected from the group consisting of an acetoxy group, a hydroxy group, an alkoxy group, and a halogen group.

In X₁ and X₂ of Formulae 3 to 5 above, the halogen group may be at least one selected from the group consisting of a chlorine atom (Cl), a bromine atom (Br), and an iodine atom (I), and the alkoxy group may be an alkoxy group having 1 to 5 carbon atoms.

Specifically, a complex according to an embodiment of the present disclosure may include at least one of compounds represented by Formulae 3a, 3b, 4a, and 5a below, in which a compound represented by any one of Formulae 1a, 1b, 1c, and 2a above is coordinately bonded to palladium or cobalt of a metal precursor compound.

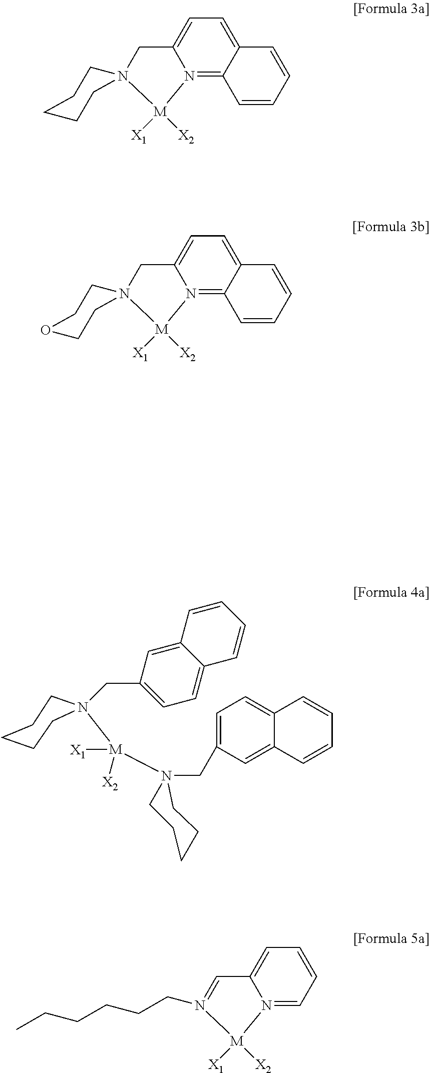

In Formulae 3a, 3b, 4a, and 5a above, M, X₁, and X₂ are substantially the same as M, X₁, and X₂ described in Formulae 3 to 5.

The complex represented by Formulae 3 to 5 above may be prepared by reacting an amine compound represented by Formulae 1 and 2 with a palladium or cobalt-containing metal precursor, and then used as a catalyst in the oligomerization reaction or polymerization reaction of the cyclic olefin-based monomer together with a cocatalyst compound which will be described below. Here, the reaction of the amine compound and the metal precursor compound may be carried out at room temperature, preferably 10° C. to 30° C., and the reaction time may be 10 to 48 hours.

Meanwhile, in the present disclosure, the cocatalyst compound serves to activate the metal component of the metal precursor compound, and may be a non-metal compound, and preferably may be an organic boron compound.

Examples of the organic boron compound include trimethylammonium tetrakis(pentafluorophenyl)borate, triethylammonium tetrakis(pentafluorophenyl)borate, N,N-dimethylanilinium tetrakis(pentafluorophenyl)borate, N,N-dimethylanilinium n-butyltris(pentafluorophenyl)borate, N,N-dimethylanilinium benzyltris(pentafluorophenyl)borate, N,N-dimethylanilinium tetrakis(4-(t-butyldimethylsilyl)-2,3,5,6-tetrafluorophenyl)borate, N,N-dimethylanilinium tetrakis(4-(triisopropylsilyl)-2,3,5,6-tetrafluorophenyl)borate, N,N-dimethylanilinium pentafluorophenoxytris(pentafluorophenyl)borate, N,N-dimethyl-2,4,6-trimethylanilinium tetrakis(pentafluorophenyl)borate, trimethylammonium tetrakis(2,3,4,6-tetrafluorophenyl)borate, N,N-dimethylanilinium tetrakis(2,3,4,6-tetrafluorophenyl)borate, hexadecyldimethylammonium tetrakis(pentafluorophenyl)borate, N-methyl-N-dodecylanilinium tetrakis(pentafluorophenyl)borate, methyldi(dodecyl)ammonium tetrakis(pentafluorophenyl)borate, and the like. Of these, more preferred is tetrakis(pentafluorophenyl)borate.

The cocatalyst compound may be contained in an amount of 2 to 10 moles, preferably 2 to 5 moles, and more preferably 2 moles, per 1 mole of the palladium or cobalt-containing metal precursor compound. When the above content ratio is satisfied, a boron anion in the cocatalyst may interact with a cationic catalyst composition to activate the catalyst composition to coordinate with a monomer.

The catalyst composition for cyclic olefin polymerization may be added together with the cyclic olefin-based monomer during the polymerization of the desired cyclic olefin-based monomer to form a cyclic olefin-based polymer or a cyclic olefin-based oligomer. During the oligomerization reaction or polymerization reaction of the cyclic olefin-based monomer, the N position of the amine compound of Formula 1 or 2 may be instantly coordinated to the palladium atom or cobalt atom of the metal precursor compound to form a complex.

At this time, the catalyst composition for cyclic olefin polymerization may be added during the polymerization of cyclic olefin-based monomer in a state in which each component of the catalyst composition is dissolved in an inert solvent. Non-limiting examples of the inert solvent include: an aromatic hydrocarbon such as benzene, toluene, xylene, benzonitrile, nitrobenzene, adiponitrile, anisole, and phenylnonane; an aliphatic hydrocarbon having 5 to 20 carbon atoms such as pentane, hexane, heptane, and adiponitrile; a halogenated hydrocarbon such as methylene chloride, 1,2-dichloroethane, chloroform, and carbon tetrachloride; a non-fluorinated saturated substituted aliphatic hydrocarbon and/or aromatic hydrocarbon having 1 to 20 carbon atoms, including a compound selected from the group consisting of alcohols such as methanol, propanol, butanol, isopropanol, and 2,4-ditert-butyl phenol; a ketone such as acetone; a carboxylic acid such as propanoic acid and acetic acid; an ester such as ethyl acetate, ethyl benzoate, dimethyl succinate, butyl acetate, tri-n-butyl phosphate, and dimethyl phosphate; an ether such as tetraethylene glycol dimethyl ether (tetraglyme); and mixtures thereof. Of these, preferred is methylene chloride.

Another aspect of the present disclosure provides a method for preparing a cyclic olefin-based oligomer, the method including subjecting a cyclic olefin-based monomer to an oligomerization reaction in the presence of the above-described catalyst composition for cyclic olefin polymerization.

As the cyclic olefin-based monomer, any cyclic olefin-based monomer capable of forming an oligomer may be used. As an example, the cyclic olefin-based monomer may include at least one selected from the group consisting of norbornene (NB), dicyclopentadiene (DCPD), cyclopentadiene (CPD), cyclopentene (Cp), cyclobutene (Cb), cyclohexene (Chx), cycloheptene (Chp), cyclooctene (Cot), and derivatives thereof. Preferably, the cyclic olefin-based monomer may be a compound represented by Formula 10 below.

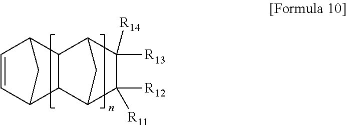

In Formula 10 above, n is an integer of 0 to 4, and R₁₁ to R₁₄ are the same or different and are each independently selected from the group consisting of: a hydrogen atom; a halogen atom; a hydroxyl group; a carboxyl group; a straight or branched alkyl group having 1 to 20 carbon atoms; a cycloalkyl group having 3 to 12 carbon atoms; an aryl group having 6 to 20 carbon atoms; an alkoxy group having 1 to 10 carbon atoms; and an acyl group having 1 to 10 carbon atoms.

In Formula 10 above, n is an integer of 0 to 2, and R₁₁ to R₁₄ are the same or different and are each independently selected from the group consisting of: a hydrogen atom; a carboxyl group; a straight or branched alkyl group having 1 to 5 carbon atoms; a cycloalkyl group having 3 to 12 carbon atoms; an aryl group having 6 to 18 carbon atoms; an alkoxy group having 1 to 5 carbon atoms; and an acyl group having 1 to 6 carbon atoms, which is preferable in terms of polymerization.

In the present disclosure, the cyclic olefin-based monomer described above may be homopolymerized, two or more cyclic olefin-based monomers may be copolymerized, or the cyclic olefin-based monomer and an olefin-based monomer may be copolymerized. In this case, any cyclic olefin-based monomer capable of undergoing oligomerization with the cyclic olefin-based monomer may be used without limitation. In terms of the physical properties of the prepared oligomer, the cyclic olefin-based monomer may be a monomer containing a polar vinyl group such as vinyl acetate, acrylate, alkyl methacrylate, and methyl methacrylate.

In the case of polymerizing the cyclic olefin-based monomer using the catalyst composition for cyclic olefin polymerization according to the present disclosure, the reaction may be performed in a slurry phase, a liquid phase, or a gas phase. When performing the reaction in the liquid or slurry phase, a solvent or olefin itself may be used as a medium. The solvent used herein may include at least one solvent selected from the group consisting of methylene chloride, 1,2-dichlorobenzene, toluene, n-pentane, n-hexane, n-heptane, chlorobenzene, dichloromethane, chloroform, 1,2-dichloroethane, and 1,1,2,2-tetrachloroethane.

In this case, the cyclic olefin-based monomer may be reacted by mixing 500 to 15,000 moles of the cyclic olefin-based monomer with respect to 1 mole of the palladium or cobalt-containing metal precursor compound of the catalyst composition, and preferably 1,000 to 10,000 moles. When the cyclic olefin-based monomer is used in an amount less than 500 moles with respect to 1 mole of the palladium or cobalt-containing metal precursor compound, it may be economically disadvantageous due to excessive use of the catalyst, and when it is used in an amount exceeding 15,000 moles, the polymerization yield may be reduced due to a decrease in the amount of the catalyst composition used compared to the cyclic olefin-based monomer.

The reaction may be performed in a batch, semi-continuous, or continuous manner. The reaction may be performed under the condition of a temperature of −30° C. to 120° C., preferably at room temperature, for 1 to 26 hours. When performing the reaction at a temperature below −30° C. or for less than 1 hour, the reaction may insufficiently proceed. On the contrary, when performing the reaction at a temperature exceeding 120° C. or for over 26 hours, the polymer chain may be decomposed, thereby decreasing molecular weight or causing gelation.

The cyclic olefin-based oligomer prepared in this manner may have a very low amount of catalyst residue in the oligomer, so a separate purification process may not be required, and may have stereoselectivity that is not achieved by free radical polymerization, a high molecular weight, or an appropriate glass transition temperature that allows for molding processability.

In addition, another aspect of the present disclosure provides a method for preparing a cyclic olefin-based polymer, the method including performing addition polymerization of a cyclic olefin-based monomer in the presence of the above-described catalyst composition for cyclic olefin polymerization. The specific steps and reaction conditions thereof are also replaced with those described above in the cyclic olefin oligomerization reaction.

Hereinafter, the present disclosure will be specifically described according to examples, but the present disclosure is not limited by the following examples.

### Preparation Example 1: Preparation of 2-(piperidine-1-ylmethyl)quinoline (L1)

2-(piperidine-1-ylmethyl)quinoline (hereinafter, L1) was prepared by the following method with reference to “Tetrahedron Letters 2018, 59, 1723”.

4.28 g (20.0 mmol) of 2-(chloromethyl)quinoline hydrochloride was dissolved in 50 mL of distilled water, and 1.70 g (20.0 mmol) of piperidine was added and mixed therewith. 2.24 g (40.0 mmol) of KOH was gradually added to the resulting mixture and reacted at room temperature for 24 hours to obtain a reaction product. After the obtained reaction product was extracted with 50 mL of methylene chloride, the residual water was removed with anhydrous MgSO₄ and filtered. A solvent obtained by filtration was removed by drying under reduced pressure. The resulting mixture was distilled under reduced pressure to prepare 2.63 g (58.0%) of 2-(piperidine-1-ylmethyl)quinoline as a yellow solid compound represented by Formula 1a.

### Preparation Example 2: Preparation of 4-(quinolin-2-ylmethyl)morpholine (L2)

4-(quinolin-2-ylmethyl)morpholine (hereinafter, L2) was prepared by the following method with reference to “Tetrahedron Letters 2018, 59, 1723”.

4.67 g (21.8 mmol) of 2-(chloromethyl)quinoline hydrochloride was dissolved in 50 mL of distilled water, and 1.90 g (21.8 mmol) of morpholine was added and mixed therewith. 2.45 g (43.6 mmol) of KOH was gradually added to the resulting mixture and reacted at room temperature for 24 hours to obtain a reaction product. After the obtained reaction product was extracted with 50 mL of methylene chloride, the residual water was removed with anhydrous MgSO₄ and filtered. A solvent obtained by filtration was removed by drying under reduced pressure. The resulting mixture was distilled under reduced pressure to prepare 2.84 g (57.0%) of 4-(quinolin-2-ylmethyl)morpholine as a yellow, highly viscous liquid compound represented by Formula 1b.

### Preparation Example 3: Preparation of (E)-N-(pyridin-2-ylmethylene)hexan-1-amine (L3)

4-(quinolin-2-ylmethyl)morpholine (hereinafter, L3) was prepared by the following method with reference to “Inorganica Chimica Acta, 2022, 539, 121025”.

2.02 g (20.0 mmol) of hexylamine was dissolved in 50 mL of methylene chloride, and 2.14 g (20.0 mmol) of 2-pyridinecarboxaldehyde was added and mixed therewith. The resulting mixture was reacted at room temperature for 72 hours to obtain a reaction product. After the obtained reaction product was extracted with methylene chloride, the residual water was removed with anhydrous MgSO₄ and filtered. A solvent obtained by filtration was removed by drying under reduced pressure. The resulting mixture was distilled under reduced pressure to prepare 1.51 g (39.8%) of (E)-N-(pyridin-2-ylmethylene)hexan-1-amine as a fluorescent yellow liquid compound represented by Formula 2a.

### Preparation Example 4: Preparation of (E)-N-(pyridin-2-ylmethylene)hexan-1-amine (L4)

3.53 g (20.0 mmol) of 2-(chloromethyl)naphthalene was dissolved in 25 mL of methylene chloride, and 1.70 g (20.0 mmol) of piperidine was added and mixed therewith. 1.68 g (30.0 mmol) of KOH and 10 mL of distilled water were gradually added to the resulting mixture and reacted at room temperature for 4 days to obtain a reaction product. After the obtained reaction product was extracted with methylene chloride, the residual water was removed with anhydrous MgSO₄ and filtered. A solvent obtained by filtration was removed by drying under reduced pressure. The resulting mixture was distilled under reduced pressure to prepare 3.01 g (66.5%) of 1-(naphthalen-2-ylmethyl)piperidine as a yellow solid compound represented by Formula 1c.

1H NMR (500 MHz; CDCl₃): 57.84-7.80 (3H, m, Naphthalene-H), 7.75 (1H, s, Naphthalene-H), 7.52 (1H, dd, J=8.45 Hz, Naphthalene-H), 7.46 (2H, m, Naphthalene-H), 3.65 (2H, s, NPiperidine-CH₂-Naphthalene), 2.45 (4H, s, Piperidine-H), 1.61 (4H, quin, J=11.23 Hz, Piperidine-H), 1.46 (2H, m, Piperidine-H)

### Preparation Example 5: Preparation of Palladium(II) Acetate Complex Using L1

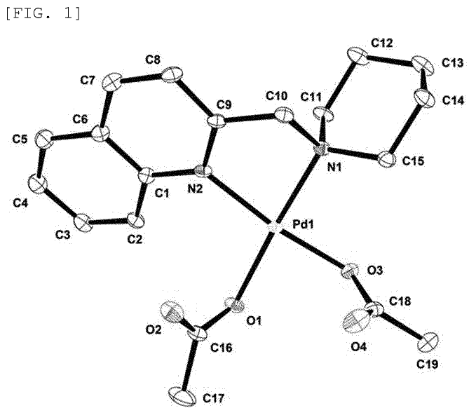

0.23 g (1.0 mmol) of an amine compound L1 prepared in Preparation Example 1 and 0.22 g (1.0 mmol) of palladium (II) acetate were each dissolved in 10 mL of anhydrous methylene chloride, and the amine compound solution was gradually added to the palladium (II) acetate solution. The resulting mixture was stirred for 24 hours and then dried in vacuum, leaving only about 5 mL of methylene chloride. 10 mL of anhydrous hexane was gradually added to the remaining solution and left to rest until the layer disappeared. The resulting powder was then washed with anhydrous hexane four or more times. After washing, the resulting product was dried in vacuum for 18 hours to prepare 0.31 g (68.8%) of a complex (PdL1(OAc)₂) as a light green powder represented by Formula 3a (M: Pd, X₁ and X₂: acetoxy group). The structure of the prepared complex is illustrated in FIG. 1.

¹H NMR (500 MHz; CDCl₃): 58.82 (1H, d, J=8.91 Hz, Quinoline-H), 8.34 (1H, d, J=8.28 Hz, Quinoline-H), 7.81 (1H, d, J=8.21 Hz, Quinoline-H), 7.78 (1H, m, Quinoline-H), 7.63 (1H, m, Quinoline-H), 7.42 (1H, d, J=8.35 Hz, Quinoline-H), 4.74 (2H, s, NPiperidine-CH₂-Quinoline), 3.46 (2H, m, Piperidine-H), 2.71 (2H, m, Piperidine-H), 2.06 (3H, s, Palladium(II) acetate-H), 1.86 (3H, s, Palladium(II) acetate-H), 1.81-1.76 (2H, m, Piperidine-H), 1.75-1.68 (2H, m, Piperidine-H), 1.65-1.56 (2H, m, Piperidine-H)

### Preparation Example 6: Preparation of Palladium(II) Acetate Complex Using L4

0.23 g (1.0 mmol) of an amine compound L4 prepared in Preparation Example 4 and 0.11 g (0.5 mmol) of palladium (II) acetate were each dissolved in 10 mL of anhydrous methylene chloride, and the amine compound solution was gradually added to the palladium (II) acetate solution. The resulting mixture was stirred for 24 hours and then dried in vacuum, leaving only about 5 mL of methylene chloride. 10 mL of anhydrous hexane was gradually added to the remaining solution and left to rest until the layer disappeared. The resulting powder was then washed with anhydrous hexane four or more times. After washing, the resulting product was dried in vacuum for 18 hours to prepare 0.11 g (32.6%) of a complex (Pd(L4)₂(OAc)₂) as a light green powder represented by Formula 4a (M: Pd, X₁ and X₂: acetoxy group).

¹H NMR (500 MHz; CDCl₃): 57.68 (5H, m, Quinoline-H), 7.36-7.31 (9H, m, Quinoline-H), 3.65 (2H, m, NPiperidine-CH₂-Quinoline), 3.56 (2H, m, NPiperidine-CH₂-Quinoline), 3.51 (2H, m, Piperidine-H), 2.80-2.78 (2H, m, Piperidine-H), 2.74 (2H, m, Piperidine-H), 2.17 (6H, s, Palladium(II) acetate-H), 2.09-2.05 (2H, m, Piperidine-H), 1.68-1.60 (4H, m, Piperidine-H), 1.42-1.37 (2H, m, Piperidine-H), 1.31-1.28 (2H, m, Piperidine-H), 1.19-1.16 (2H, m, Piperidine-H), 1.10-1.04 (2H, m, Piperidine-H)

### Preparation Example 7: Preparation of Palladium(II) Acetate Complex Using L4

A complex was prepared in the same manner as in Preparation Example 6, except that 0.23 g (1.0 mmol) of L4 and 0.22 g (1.0 mmol) of palladium (II) acetate were used. As a result, 0.23 g (68.1%) of a complex as a light green powder represented by Formula 4a (M: Pd, X₁ and X₂: acetoxy group) was obtained.

¹H NMR (500 MHz; CDCl₃): 57.68 (5H, m, Quinoline-H), 7.36-7.31 (9H, m, Quinoline-H), 3.65 (2H, m, NPiperidine-CH₂-Quinoline), 3.56 (2H, m, NPiperidine-CH₂-Quinoline), 3.51 (2H, m, Piperidine-H), 2.80-2.78 (2H, m, Piperidine-H), 2.74 (2H, m, Piperidine-H), 2.17 (6H, s, Palladium(II) acetate-H), 2.09-2.05 (2H, m, Piperidine-H), 1.68-1.60 (4H, m, Piperidine-H), 1.42-1.37 (2H, m, Piperidine-H), 1.31-1.28 (2H, m, Piperidine-H), 1.19-1.16 (2H, m, Piperidine-H), 1.10-1.04 (2H, m, Piperidine-H)

### Examples 1 to 6: In-Situ Preparation of Norbornene Polymer

### Example 1: Polymerization Reaction Using Catalyst Composition Containing Amine Compound L1

First, as illustrated in Table 1 below, 6.8 mg of an amine compound L1 prepared in Preparation Example 1, 6.7 mg (30.0 μmol) of palladium (II) acetate as a metal precursor compound, and 48.1 mg (60.0 μmol) of N,N-dimethylanilinium tetrakis(fluorophenyl)borate as a cocatalyst compound were put into a 50 mL Schlenk flask under an argon atmosphere, and they were dissolved by adding 20 mL of methylene chloride. The flask was stirred for 1 hour to activate the catalyst stock solution. 1.41 g (15.0 mmol) of norbornene was put into another 100 mL Schlenk flask under an argon atmosphere, and dissolved by adding 20 mL of chlorobenzene. Then, 0.1 mL (0.75 mmol) of tetralin was added as a reference substance to measure the conversion rate. 10 mL of the activated catalyst composition was added thereto, and a polymerization reaction was performed at 25° C. for 2 hours. After the reaction, the resulting mixture was diluted by adding 40 mL of dimethoxypropane, and 10 mL of hexane was gradually added to precipitate a reaction product. The resulting mixture was added to 200 mL of hexane, stirred for 30 minutes, and filtered through a reduced pressure filter to recover a reaction product. The reaction product was dried in a vacuum oven at 70° C. to prepare a norbornene polymer. The polymerization results were measured by NMR. The measurement results are illustrated in Table 2 as the yield and the conversion rate of the norbornene polymer.

### Example 2: Polymerization Reaction Using Catalyst Composition Containing Amine Compound L1

A norbornene polymer was prepared in the same manner as in Example 1, except that 2 mL of a catalyst composition was added to a monomer flask instead of 10 mL of a catalyst composition as described in Table 1 below, and the molar ratio of the catalyst composition and the monomer was changed to 1:5,000 to prepare a norbornene polymer. The polymerization results were measured by NMR. The measurement results are illustrated in Table 2 as the yield and the conversion rate of the norbornene polymer.

### Example 3: Polymerization Reaction Using Catalyst Composition Containing Amine Compound L1

A norbornene polymer was prepared in the same manner as in Example 1, except that 1 mL of a catalyst composition was added to a monomer flask instead of 10 mL of a catalyst composition as described in Table 1 below, and the molar ratio of the catalyst composition and the monomer was changed to 1:10,000 to prepare a norbornene polymer. The polymerization results were measured by NMR. The measurement results are illustrated in Table 2 as the yield and the conversion rate of the norbornene polymer.

### Example 4: Polymerization Reaction Using Catalyst Composition Containing Amine Compound L4

A norbornene polymer was prepared in the same manner as in Example 1, except that 6.8 mg of an amine compound L4 prepared in Preparation Example 4 was added instead of an amine compound L1 as described in Table 1 below to prepare a norbornene polymer. The polymerization results were measured by NMR. The measurement results are illustrated in Table 2 as the yield and the conversion rate of the norbornene polymer.

### Example 5: Polymerization Reaction Using Catalyst Composition Containing Amine Compound L4

A norbornene polymer was prepared in the same manner as in Example 2, except that 6.8 mg of an amine compound L4 prepared in Preparation Example 4 was added instead of an amine compound L1 as described in Table 1 below to prepare a norbornene polymer. The polymerization results were measured by NMR. The measurement results are illustrated in Table 2 as the yield and the conversion rate of the norbornene polymer.

### Example 6: Polymerization Reaction Using Catalyst Composition Containing Amine Compound L4

A norbornene polymer was prepared in the same manner as in Example 3, except that 6.8 mg of an amine compound L4 prepared in Preparation Example 4 was added instead of an amine compound L1 as described in Table 1 below to prepare a norbornene polymer. The polymerization results were measured by NMR. The measurement results are illustrated in Table 2 as the yield and the conversion rate of the norbornene polymer.

### Examples 7 to 12: Preparation of Norbornene Oligomer

### Example 7: Oligomerization Reaction Using Catalyst Composition Containing Amine Compound L2

A norbornene polymer was prepared in the same manner as in Example 1, except that 6.8 mg of an amine compound L2 prepared in Preparation Example 2 was added instead of an amine compound L1 as described in Table 1 below to prepare a norbornene oligomer. The polymerization results were measured by NMR. The measurement results are illustrated in Table 2 as the yield and the conversion rate of the norbornene oligomer.

### Example 8: Oligomerization Reaction Using Catalyst Composition Containing Amine Compound L2

A norbornene polymer was prepared in the same manner as in Example 2, except that 6.8 mg of an amine compound L2 prepared in Preparation Example 2 was added instead of an amine compound L1 as described in Table 1 below to prepare a norbornene oligomer. The polymerization results were measured by NMR. The measurement results are illustrated in Table 2 as the yield and the conversion rate of the norbornene oligomer.

### Example 9: Oligomerization Reaction Using Catalyst Composition Containing Amine Compound L2

A norbornene polymer was prepared in the same manner as in Example 3, except that 6.8 mg of an amine compound L2 prepared in Preparation Example 2 was added instead of an amine compound L1 as described in Table 1 below to prepare a norbornene oligomer. The polymerization results were measured by NMR. The measurement results are illustrated in Table 2 as the yield and the conversion rate of the norbornene oligomer.

### Example 10: Oligomerization Reaction Using Catalyst Composition Containing Amine Compound L3

A norbornene polymer was prepared in the same manner as in Example 1, except that 5.7 mg of an amine compound L3 prepared in Preparation Example 3 was added instead of an amine compound L1 as described in Table 1 below to prepare a norbornene oligomer. The polymerization results were measured by NMR. The measurement results are illustrated in Table 2 as the yield and the conversion rate of the norbornene oligomer.

### Example 11: Oligomerization Reaction Using Catalyst Composition Containing Amine Compound L3

A norbornene polymer was prepared in the same manner as in Example 2, except that 5.7 mg of an amine compound L3 prepared in Preparation Example 3 was added instead of an amine compound L1 as described in Table 1 below to prepare a norbornene oligomer. The polymerization results were measured by NMR. The measurement results are illustrated in Table 2 as the yield and the conversion rate of the norbornene oligomer.

### Example 12: Oligomerization Reaction Using Catalyst Composition Containing Amine Compound L3

A norbornene polymer was prepared in the same manner as in Example 3, except that 5.7 mg of an amine compound L3 prepared in Preparation Example 3 was added instead of an amine compound L1 as described in Table 1 below to prepare a norbornene oligomer. The polymerization results were measured by NMR. The measurement results are illustrated in Table 2 as the yield and the conversion rate of the norbornene oligomer.

TABLE 1

| | | Catalyst composition | |
|---|---|---|---|
| | | (molar ratio) | Monomer:metal |
| | | Metal precursor | precursor |
| | | compound:amine | compound |
| | Amine | compound:cocatalyst | (molar |
| Classification | compound | compound | ratio) |
| Example 1 | L1 | 1:1:2 | 1,000:1 |
| Example 2 | L1 | 1:1:2 | 5,000:1 |
| Example 3 | L1 | 1:1:2 | 10,000:1 |
| Example 4 | L4 | 1:1:2 | 1,000:1 |
| Example 5 | L4 | 1:1:2 | 5,000:1 |
| Example 6 | L4 | 1:1:2 | 10,000:1 |
| Example 7 | L2 | 1:1:2 | 1,000:1 |
| Example 8 | L2 | 1:1:2 | 5,000:1 |
| Example 9 | L2 | 1:1:2 | 10,000:1 |
| Example 10 | L3 | 1:1:2 | 1,000:1 |
| Example 11 | L3 | 1:1:2 | 5,000:1 |
| Example 12 | L3 | 1:1:2 | 10,000:1 |

TABLE 2

| Classification | Polymer yield (%) | Conversion rate (%) |
|---|---|---|
| Example 1 | 51.6 | 83.9 |
| Example 2 | 25.6 | 54.3 |
| Example 3 | 15.7 | 42.2 |
| Example 4 | 81.4 | 98.2 |
| Example 5 | 64.9 | 78.6 |
| Example 6 | 29.3 | 41.8 |
| Example 7 | — | 2.9 |
| Example 8 | — | 0.8 |
| Example 9 | — | 0.3 |
| Example 10 | — | 78.1 |
| Example 11 | — | 29.3 |
| Example 12 | — | 16.3 |

As illustrated in Table 2, it was confirmed that in the cases of Examples 1 to 12, norbornene could be polymerized at a high conversion rate in situ using the catalyst composition, and in particular, in the case of Example 4, a high yield polymer could be prepared at a high conversion rate.

### Examples 13 and 14: Ex-Situ Preparation of Norbornene Polymer

### Example 13: Polymerization Reaction Using Palladium(II) Acetate Complex of L1

First, as illustrated in Table 3 below, 10.1 mg (22.5 μmol) of palladium(II) acetate complex of L1 prepared in Preparation Example 5 and 36.1 mg (45.0 μmol) of N,N-dimethylanilinium tetrakis(fluorophenyl)borate as a cocatalyst compound were put into a 50 mL Schlenk flask under an argon atmosphere, and they were dissolved by adding 15 mL of methylene chloride. The flask was stirred for 1 hour to activate the catalyst stock solution. 1.41 g (15.0 mmol) of norbornene was put into another 100 mL Schlenk flask under an argon atmosphere, and dissolved by adding 20 mL of chlorobenzene. Then, 0.1 mL (0.75 mmol) of tetralin was added as a reference substance to measure the conversion rate. 10 mL of the activated catalyst composition was added thereto, and a polymerization reaction was performed at 25° C. for 2 hours. After the reaction, the resulting mixture was diluted by adding 40 mL of dimethoxypropane, and 10 mL of hexane was gradually added to precipitate a reaction product. The resulting mixture was added to 200 mL of hexane, stirred for 30 minutes, and filtered through a reduced pressure filter to recover a reaction product. The reaction product was dried in a vacuum oven at 70° C. to prepare a norbornene polymer. The polymerization results were measured by NMR. The measurement results are illustrated in Table 4 as the yield and the conversion rate of the norbornene polymer.

### Example 14: Polymerization Reaction Using Palladium(II) Acetate Complex of L4

A norbornene polymer was prepared in the same manner as in Example 13, except that 15.2 mg of a palladium (II) acetate complex of L4 prepared in Preparation Example 6 was added instead of a palladium (II) acetate complex of L1 as described in Table 3 below to prepare a norbornene polymer. The polymerization results were measured by NMR. The measurement results are illustrated in Table 4 as the yield and the conversion rate of the norbornene polymer.

TABLE 3

| | | Catalyst | |
|---|---|---|---|
| | | composition | |
| | | (molar ratio) | Monomer:palladium |
| | | Palladium | complex |
| Classifi- | Palladium | complex:cocatalyst | (molar |
| cation | complex | compound | ratio) |
| Example 13 | PdL1(OAc)₂ | 1:2 | 1,000:1 |
| Example 14 | Pd(L4)₂(OAc)₂ | 1:2 | 1,000:1 |

TABLE 4

| Classification | Polymer yield (%) | Conversion rate (%) |
|---|---|---|
| Example 13 | 67.8 | 75.9 |
| Example 14 | 69.6 | 89.6 |

As illustrated in Table 4, it was confirmed that in the cases of in Examples 13 and 14 using the complex, norbornene could be polymerized at a high conversion rate.

### Examples 15 and 16: In-Situ Preparation of Butylnorbornene Oligomer

### Example 15: Oligomerization Reaction Using Catalyst Composition Containing Amine Compound L1

First, as illustrated in Table 5 below, 6.8 mg of an amine compound L1 prepared in Preparation Example 1, 6.7 mg (30.0 μmol) of palladium (II) acetate as a metal precursor compound, and 48.1 mg (60.0 μmol) of N,N-dimethylanilinium tetrakis(fluorophenyl)borate as a cocatalyst compound were put into a 50 mL Schlenk flask under an argon atmosphere, and they were dissolved by adding 20 mL of methylene chloride. The flask was stirred for 1 hour to activate the catalyst stock solution. 2.5 mL (15.0 mmol) of butylnorbornene (exo-isomer:endo-isomer=25:75) was put into another 100 mL Schlenk flask under an argon atmosphere, and dissolved by adding 20 mL of chlorobenzene. Then, 0.1 mL (0.75 mmol) of tetralin was added as a reference substance to measure the conversion rate. 10 mL of the activated catalyst composition was added thereto, and an oligomerization reaction was performed at 25° C. for 2 hours. After the reaction, the resulting mixture was diluted by adding 40 mL of dimethoxypropane, and 10 mL of hexane was gradually added to precipitate a reaction product. The resulting mixture was added to 200 mL of hexane, stirred for 30 minutes, and filtered through a reduced pressure filter to recover an oligomer. The oligomer was dried in a vacuum oven at 70° C. to finally prepare a butylnorbornene oligomer. The polymerization results were measured by NMR. The measurement results are illustrated in Table 6 as the yield and the conversion rate of the butylnorbornene oligomer.

### Example 16: Oligomerization Reaction Using Catalyst Composition Containing Amine Compound L4

A butylnorbornene oligomer was prepared in the same manner as in Example 15, except that 6.8 mg of an amine compound L4 prepared in Preparation Example 4 was added instead of an amine compound L1 as described in Table 5 below to prepare a butylnorbornene oligomer. The polymerization results were measured by NMR. The measurement results are illustrated in Table 6 as the yield and the conversion rate of the butylnorbornene oligomer.

TABLE 5

| | | Catalyst composition | |
|---|---|---|---|
| | | (molar ratio) | |
| | | Metal precursor | Monomer:metal |
| | | compound:amine | precursor |
| | Amine | compound:cocatalyst | compound |
| Classification | compound | compound | (molar ratio) |
| Example 15 | L1 | 1:1:2 | 1,000:1 |
| Example 16 | L4 | 1:1:2 | 1,000:1 |

TABLE 6

Conversion rate (%)

| Classification | Exo type | Endo type |
|---|---|---|
| Example 15 | 31.0 | 16.8 |
| Example 16 | 90.0 | 52.9 |

As illustrated in Table 6, it was confirmed that in the cases of Examples 15 and 16, a butylnorbornene oligomer could be polymerized at a high conversion rate in situ using the catalyst composition.

### Examples 17 and 18: In-Situ Preparation of Methylnorbornene Oligomer

### Example 17: Oligomerization Reaction Using Catalyst Composition Containing Amine Compound L1

First, as illustrated in Table 7 below, 6.8 mg of an amine compound L1 prepared in Preparation Example 1, 6.7 mg (30.0 μmol) of palladium (II) acetate as a metal precursor compound, and 48.1 mg (60.0 μmol) of N,N-dimethylanilinium tetrakis(fluorophenyl)borate as a cocatalyst compound were put into a 50 mL Schlenk flask under an argon atmosphere, and they were dissolved by adding 20 mL of methylene chloride. The flask was stirred for 1 hour to activate the catalyst stock solution. 2.2 mL (15.0 mmol) of methylnorbornene (exo-isomer:endo-isomer=45:55) was put into another 100 mL Schlenk flask under an argon atmosphere, and dissolved by adding 20 mL of chlorobenzene. Then, 0.1 mL (0.75 mmol) of tetralin was added as a reference substance to measure the conversion rate. 10 mL of the activated catalyst composition was added thereto, and an oligomerization reaction was performed at 25° C. for 2 hours. After the reaction, the resulting mixture was diluted by adding 40 mL of dimethoxypropane, and 10 mL of hexane was gradually added to precipitate a reaction product. The resulting mixture was added to 200 mL of hexane, stirred for 30 minutes, and filtered through a reduced pressure filter to recover an oligomer. The oligomer was dried in a vacuum oven at 70° C. to finally prepare a methylnorbornene oligomer. The polymerization results were measured by NMR. The measurement results are illustrated in Table 8 as the yield and the conversion rate of the methylnorbornene oligomer.

### Example 18: Oligomerization Reaction Using Catalyst Composition Containing Amine Compound L4

A methylnorbornene oligomer was prepared in the same manner as in Example 17, except that 6.8 mg of an amine compound L4 prepared in Preparation Example 4 was added instead of an amine compound L1 as described in Table 7 below to prepare a methylnorbornene oligomer. The polymerization results were measured by NMR. The measurement results are illustrated in Table 8 as the yield and the conversion rate of the methylnorbornene oligomer.

TABLE 7

| | | Catalyst composition | |
|---|---|---|---|
| | | (molar ratio) | |
| | | Metal precursor | Monomer:metal |
| | | compound:amine | precursor |
| | Amine | compound:cocatalyst | compound |
| Classification | compound | compound | (molar ratio) |
| Example 17 | L1 | 1:1:2 | 1,000:1 |
| Example 18 | L4 | 1:1:2 | 1,000:1 |

TABLE 8

Conversion rate (%)

| Classification | Exo type | Endo type |
|---|---|---|
| Example 17 | 0.8 | 0 |
| Example 18 | 1.6 | 1.3 |

As illustrated in Table 8, it was confirmed that in the cases of Examples 17 and 18, a methylnorbornene oligomer could be polymerized in situ using the catalyst composition.

### Examples 19 and 20: In-Situ Preparation of Acetoxymethylnorbornene Oligomer

### Example 19: Oligomerization Reaction Using Catalyst Composition Containing Amine Compound L4

First, as illustrated in Table 9 below, 5.1 mg of an amine compound L4 prepared in Preparation Example 4, 5.1 mg (22.5 μmol) of palladium (II) acetate as a metal precursor compound, and 36.1 mg (45.0 μmol) of N,N-dimethylanilinium tetrakis(fluorophenyl)borate as a cocatalyst compound were put into a 50 mL Schlenk flask under an argon atmosphere, and they were dissolved by adding 15 mL of methylene chloride. The flask was stirred for 1 hour to activate the catalyst stock solution. 2.3 mL (15.0 mmol) of acetoxymethylnorbornene (exo-isomer:endo-isomer=30:70) was put into another 100 mL Schlenk flask under an argon atmosphere, and dissolved by adding 20 mL of chlorobenzene. Then, 0.1 mL (0.75 mmol) of tetralin was added as a reference substance to measure the conversion rate. 10 mL of the activated catalyst composition was added thereto, and an oligomerization reaction was performed at 25° C. for 2 hours. After the reaction, the resulting mixture was diluted by adding 40 mL of dimethoxypropane, and 10 mL of hexane was gradually added to precipitate a reaction product. The resulting mixture was added to 200 mL of hexane, stirred for 30 minutes, and filtered through a reduced pressure filter to recover an oligomer. The oligomer was dried in a vacuum oven at 70° C. to finally prepare an acetoxymethylnorbornene oligomer. The polymerization results were measured by NMR. The measurement results are illustrated in Table 10 as the yield and the conversion rate of the acetoxymethylnorbornene oligomer.

### Example 20: Oligomerization Reaction Using Catalyst Composition Containing Amine Compound L4

An acetoxymethylnorbornene oligomer was prepared in the same manner as in Example 19, except that 6.8 mg of an amine compound L4 prepared in Preparation Example 4 was added instead of an amine compound L1 as described in Table 9 below to prepare a butylnorbornene oligomer. The polymerization results were measured by NMR. The measurement results are illustrated in Table 10 as the yield and the conversion rate of the acetoxymethylnorbornene oligomer.

TABLE 9

| | | Catalyst composition | |
|---|---|---|---|
| | | (molar ratio) | |
| | | Metal precursor | Monomer:metal |
| | | compound:amine | precursor |
| | Amine | compound:cocatalyst | compound |
| Classification | compound | compound | (molar ratio) |
| Example 19 | L1 | 1:1:2 | 1,000:1 |
| Example 20 | L4 | 1:1:2 | 1,000:1 |

TABLE 10

Conversion rate (%)

| Classification | Exo type | Endo type |
|---|---|---|
| Example 19 | 1.6 | 0 |
| Example 20 | 11.5 | 3.2 |

As illustrated in Table 10, it was confirmed that in the cases of Examples 19 and 20, an acetoxymethylnorbornene oligomer could be polymerized in situ using the catalyst composition.

### Examples 21 to 24: In-Situ Preparation of Copolymer

### Example 21: Copolymerization Reaction of Norbornene and Butylnorbornene Using Catalyst Composition Containing Amine Compound L4

First, as illustrated in Table 11 below, 5.1 mg of an amine compound L4 prepared in Preparation Example 4, 5.1 mg (22.5 μmol) of palladium (II) acetate as a metal precursor compound, and 36.1 mg (45.0 μmol) of N,N-dimethylanilinium tetrakis(fluorophenyl)borate as a cocatalyst compound were put into a 50 mL Schlenk flask under an argon atmosphere, and they were dissolved by adding 15 mL of methylene chloride. The flask was stirred for 1 hour to activate the catalyst stock solution. 0.71 g (7.5 mmol) of norbornene and 1.3 mL (7.5 mmol) of butylnorbornene (exo-isomer:endo-isomer=25:75) were put into another 100 mL Schlenk flask under an argon atmosphere, and dissolved by adding 20 mL of chlorobenzene. Then, 0.1 mL (0.75 mmol) of tetralin was added as a reference substance to measure the conversion rate. 10 mL of the activated catalyst composition was added thereto, and a copolymerization reaction was performed at 25° C. for 2 hours. After the reaction, the resulting mixture was diluted by adding 40 mL of dimethoxypropane, and 10 mL of hexane was gradually added to precipitate a reaction product. The resulting mixture was added to 200 mL of hexane, stirred for 30 minutes, and filtered through a reduced pressure filter to recover an oligomer. The oligomer was dried in a vacuum oven at 70° C. to finally prepare a copolymer oligomer of norbornene and butylnorbornene. The polymerization results were measured by NMR. The measurement results are illustrated in Table 12 as the yield and the conversion rate of the copolymer oligomer.

TABLE 11

| | | Catalyst composition | |
|---|---|---|---|
| | | (molar ratio) | |
| | | Metal precursor | Monomer:metal |
| | | compound:amine | precursor |
| | Amine | compound:cocatalyst | compound |
| Classification | compound | compound | (molar ratio) |
| Example 21 | L4 | 1:1:2 | 1,000:1 |

TABLE 12

Conversion rate (%)

Butylnorbornene

| Classification | Norbornene | Exo type | Endo type |
|---|---|---|---|
| Example 21 | 79.2 | 95.3 | 57.3 |

As illustrated in Table 12, it was confirmed that in the case of Example 21, norbornene and butylnorbornene could be copolymerized in situ using the catalyst composition.

### Example 22: Copolymerization Reaction of Norbornene and Methylnorbornene Using Catalyst Composition Containing Amine Compound L4

First, as illustrated in Table 13 below, 5.1 mg of an amine compound L4 prepared in Preparation Example 4, 5.1 mg (22.5 μmol) of palladium (II) acetate as a metal precursor compound, and 36.1 mg (45.0 mol) of N,N-dimethylanilinium tetrakis(fluorophenyl)borate as a cocatalyst compound were put into a 50 mL Schlenk flask under an argon atmosphere, and they were dissolved by adding 15 mL of methylene chloride. The flask was stirred for 1 hour to activate the catalyst stock solution. 0.71 g (7.5 mmol) of norbornene and 1.1 mL (7.5 mmol) of methylnorbornene (exo-isomer:endo-isomer=45:55) were put into another 100 mL Schlenk flask under an argon atmosphere, and dissolved by adding 20 mL of chlorobenzene. Then, 0.1 mL (0.75 mmol) of tetralin was added as a reference substance to measure the conversion rate. 10 mL of the activated catalyst composition was added thereto, and a copolymerization reaction was performed at 25° C. for 2 hours. After the reaction, the resulting mixture was diluted by adding 40 mL of dimethoxypropane, and 10 mL of hexane was gradually added to precipitate a reaction product. The resulting mixture was added to 200 mL of hexane, stirred for 30 minutes, and filtered through a reduced pressure filter to recover an oligomer. The oligomer was dried in a vacuum oven at 70° C. to finally prepare a copolymer oligomer of norbornene and methylnorbornene. The polymerization results were measured by NMR. The measurement results are illustrated in Table 14 as the yield and the conversion rate of the copolymer oligomer.

TABLE 13

| | | Catalyst composition | |
|---|---|---|---|
| | | (molar ratio) | |
| | | Metal precursor | Monomer:metal |
| | | compound:amine | precursor |
| | Amine | compound:cocatalyst | compound |
| Classification | compound | compound | (molar ratio) |
| Example 22 | L4 | 1:1:2 | 1,000:1 |

TABLE 14

Conversion rate (%)

Butylnorbornene

| Classification | Norbornene | Exo type | Endo type |
|---|---|---|---|
| Example 22 | 35.2 | 11.1 | 9.4 |

As illustrated in Table 14, it was confirmed that in the case of Example 22, norbornene and methylnorbornene could be copolymerized in situ using the catalyst composition.

### Example 23: Copolymerization Reaction of Norbornene and Acetoxymethylnorbornene Using Catalyst Composition Containing Amine Compound L4

First, as illustrated in Table 15 below, 5.1 mg of an amine compound L4 prepared in Preparation Example 4, 5.1 mg (22.5 μmol) of palladium (II) acetate as a metal precursor compound, and 36.1 mg (45.0 μmol) of N,N-dimethylanilinium tetrakis(fluorophenyl)borate as a cocatalyst compound were put into a 50 mL Schlenk flask under an argon atmosphere, and they were dissolved by adding 15 mL of methylene chloride. The flask was stirred for 1 hour to activate the catalyst stock solution. 0.71 g (7.5 mmol) of norbornene and 1.2 mL (7.5 mmol) of acetoxymethylnorbornene (exo-isomer:endo-isomer=30:70) were put into another 100 mL Schlenk flask under an argon atmosphere, and dissolved by adding 20 mL of chlorobenzene. Then, 0.1 mL (0.75 mmol) of tetralin was added as a reference substance to measure the conversion rate. 10 mL of the activated catalyst composition was added thereto, and a copolymerization reaction was performed at 25° C. for 2 hours. After the reaction, the resulting mixture was diluted by adding 40 mL of dimethoxypropane, and 10 mL of hexane was gradually added to precipitate a reaction product. The resulting mixture was added to 200 mL of hexane, stirred for 30 minutes, and filtered through a reduced pressure filter to recover a copolymer. The oligomer was dried in a vacuum oven at 70° C. to finally prepare a copolymer of norbornene and acetoxymethylnorbornene. The polymerization results were measured by NMR. The measurement results are illustrated in Table 16 as the yield and the conversion rate of the copolymer.

TABLE 15

| | | Catalyst composition | |
|---|---|---|---|
| | | (molar ratio) | |
| | | Metal precursor | Monomer:metal |
| | | compound:amine | precursor |
| | Amine | compound:cocatalyst | compound |
| Classification | compound | compound | (molar ratio) |
| Example 23 | L4 | 1:1:2 | 1,000:1 |

TABLE 16

| | | Conversion rate (%) | | |
|---|---|---|---|---|
| | Polymer | | Acetoxymethylnorbornene | |
| Classification | yield (g) | Norbornene | Exo type | Endo type |
| Example 23 | 0.37 | 88.8 | 43.1 | 15.4 |

As illustrated in Table 16, it was confirmed that in the case of Example 23, norbornene and acetoxymethylnorbornene could be copolymerized in situ using the catalyst composition.

### Example 24: Copolymerization Reaction of Butylnorbornene and Methylnorbornene Using Catalyst Composition Containing Amine Compound L4

First, as illustrated in Table 17 below, 5.1 mg of an amine compound L4 prepared in Preparation Example 4, 5.1 mg (22.5 μmol) of palladium (II) acetate as a metal precursor compound, and 36.1 mg (45.0 μmol) of N,N-dimethylanilinium tetrakis(fluorophenyl)borate as a cocatalyst compound were put into a 50 mL Schlenk flask under an argon atmosphere, and they were dissolved by adding 15 mL of methylene chloride. The flask was stirred for 1 hour to activate the catalyst stock solution. 1.3 mL (7.5 mmol) of butylnorbornene (exo-isomer:endo-isomer=25:75) and 1.1 mL (7.5 mmol) of methylnorbornene (exo-isomer:endo-isomer=45:55) were put into another 100 mL Schlenk flask under an argon atmosphere, and dissolved by adding 20 mL of chlorobenzene. Then, 0.1 mL (0.75 mmol) of tetralin was added as a reference substance to measure the conversion rate. 10 mL of the activated catalyst composition was added thereto, and an oligomerization reaction was performed at 25° C. for 2 hours. After the reaction, 4 mL of hexane was gradually added to precipitate a reaction product. The resulting mixture was added to a solution containing 200 mL of hexane and 2 mL of HCl, stirred for 30 minutes, and filtered through a reduced pressure filter to recover an oligomer. The oligomer was dried in a vacuum oven at 70° C. to finally prepare a copolymer oligomer of butylnorbornene and methylnorbornene. The polymerization results were measured by NMR. The measurement results are illustrated in Table 18 as the yield and the conversion rate of the copolymer oligomer.

TABLE 17

| | | Catalyst composition | |
|---|---|---|---|
| | | (molar ratio) | |
| | | Metal precursor | Monomer:metal |
| | | compound:amine | precursor |
| | Amine | compound:cocatalyst | compound |
| Classification | compound | compound | (molar ratio) |
| Example 24 | L4 | 1:1:2 | 1,000:1 |

TABLE 18

| | Conversion rate (%) | | | |
|---|---|---|---|---|
| | Butylnorbornene | | Methylnorbornene | |
| Classification | Exo type | Endo type | Exo type | Endo type |
| Example 24 | 12.5 | 7.1 | — | 9.1 |

As illustrated in Table 18, it was confirmed that in the case of Example 24, butylnorbornene and methylnorbornene could be copolymerized in situ using the catalyst composition, and in the case of a polymerized methylnorbornene exo-isomer, it was impossible to discern an NMR peak.

Although embodiments of the present disclosure have been described with reference to the accompanying drawings, the scope of the present disclosure is not limited to the embodiments, and it will be apparent to those skilled in the art to which the present disclosure belongs that various substitutions, modifications, and changes are possible without departing from the technical spirit and scope of the present disclosure.

1. A catalyst composition for cyclic olefin polymerization, the catalyst composition comprising:
a palladium or cobalt-containing metal precursor compound; an amine compound represented by Formula 1 or 2 below; and a cocatalyst compound, 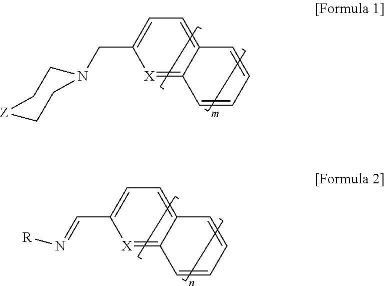
wherein in Formulae 1 and 2 above, X is a carbon atom or a nitrogen atom, Z is selected from the group consisting of an oxygen atom, a sulfur atom, and —(CH₂)—, R is a hydrogen atom or a straight or branched alkyl group having 1 to 20 carbon atoms, and m and n are the same or different and are each independently an integer of 0 to 3.

2. A catalyst composition for cyclic olefin polymerization, the catalyst composition comprising:
a complex formed by a coordination bond between a palladium or cobalt-containing metal precursor compound and an amine compound represented by Formula 1 or 2 below; and a cocatalyst compound, 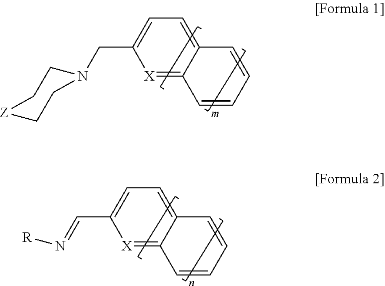
wherein in Formulae 1 and 2 above, X is a carbon atom or a nitrogen atom, Z is selected from the group consisting of an oxygen atom, a sulfur atom, and —(CH₂)—, R is a hydrogen atom or a straight or branched alkyl group having 1 to 20 carbon atoms, and m and n are the same or different and are each independently an integer of 0 to 3.

3. The catalyst composition of claim 1 or 2, wherein in Formula 2, R is a straight alkyl group having 1 to 7 carbon atoms.

4. The catalyst composition of claim 1 or 2, wherein the amine compound represented by Formula 1 above is a compound represented by any one of Formulae 1a, 1b, and 1c below,

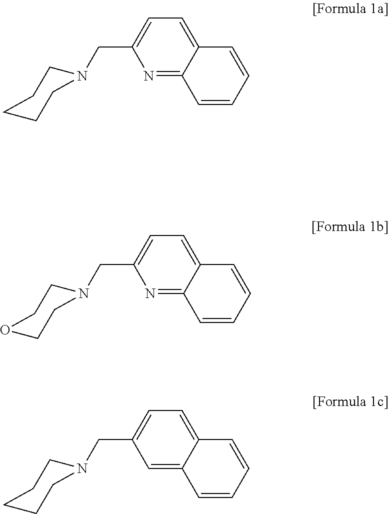 [SMILES: c1ccc2nc(CN3CCCCC3)ccc2c1 ; c1ccc2nc(CN3CCOCC3)ccc2c1 ; c1ccc2cc(CN3CCCCC3)ccc2c1]

5. The catalyst composition of claim 1 or 2, wherein the amine compound represented by Formula 2 above is a compound represented by Formula 2a below,

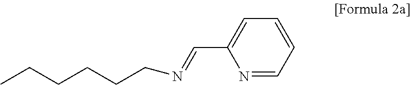

6. The catalyst composition of claim 1 or 2, wherein the cocatalyst compound is an organic boron compound.

7. The catalyst composition of claim 6, wherein the organic boron compound is tetrakis(pentafluorophenyl)borate.

8. The catalyst composition of claim 1, wherein the amine compound represented by Formula 1 or 2 above is contained in an amount of 1 to 5 moles per 1 mole of the palladium or cobalt-containing metal precursor compound.

9. The catalyst composition of claim 1, wherein the cocatalyst compound is contained in an amount of 2 to 10 moles per 1 mole of the palladium or cobalt-containing metal precursor compound.

10. The catalyst composition of claim 2, wherein the complex is a compound represented by any one of Formulae 3, 4, and 5 below, 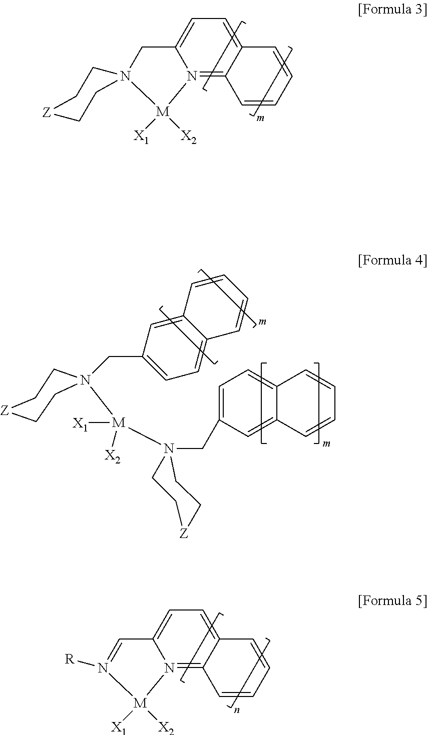
wherein in Formulae 3 to 5 above, M is palladium or cobalt, Z is the same or different and is independently selected from the group consisting of an oxygen atom, a sulfur atom, and —(CH₂)—, R is a hydrogen atom or a straight or branched alkyl group having 1 to 20 carbon atoms, X₁ and X₂ are the same or different and are each independently selected from the group consisting of an acetoxy group, a hydroxy group, an alkoxy group, and a halogen group, and m and n are the same or different and are each independently an integer of 0 to 3.

11. A method for preparing a cyclic olefin-based oligomer, the method comprising:
subjecting a cyclic olefin-based monomer to an oligomerization reaction in the presence of the catalyst composition of claim 1 or 2.

12. The method of claim 11, wherein the cyclic olefin-based monomer is a compound represented by Formula 10 below, 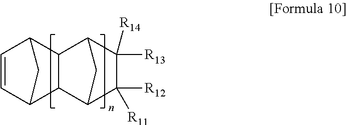
wherein in Formula 10 above, n is an integer of 0 to 4, and R₁₁ to R₁₄ are the same or different and are each independently selected from the group consisting of: a hydrogen atom; a halogen atom; a hydroxyl group; a carboxyl group; a straight or branched alkyl group having 1 to 20 carbon atoms; a cycloalkyl group having 3 to 12 carbon atoms; an aryl group having 6 to 20 carbon atoms; an alkoxy group having 1 to 10 carbon atoms; and an acyl group having 1 to 10 carbon atoms.

13. A method for preparing a cyclic olefin-based polymer, the method comprising:
performing addition polymerization of a cyclic olefin-based monomer in the presence of the catalyst composition of claim 1 or 2.

14. The method of claim 13, wherein the cyclic olefin-based monomer is a compound represented by Formula 10 below, 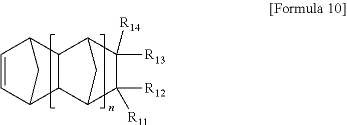
wherein in Formula 10 above, n is an integer of 0 to 4, and R₁₁ to R₁₄ are the same or different and are each independently selected from the group consisting of: a hydrogen atom; a halogen atom; a hydroxyl group; a carboxyl group; a straight or branched alkyl group having 1 to 20 carbon atoms; a cycloalkyl group having 3 to 12 carbon atoms; an aryl group having 6 to 20 carbon atoms; an alkoxy group having 1 to 10 carbon atoms; and an acyl group having 1 to 10 carbon atoms.
